(19) United States
(12) Patent Application Publication
(10) Pub. No.: US 2007/0016265 A1
(43) Pub. Date: Jan. 18, 2007

(54) Method and system for training adaptive control of limb movement

(75) Inventors: Rahman Davoodi, Glendale, CA (US); Gerald E. Loeb, South Pasadena, CA (US); Junkwan Lee, Los Angeles, CA (US)

Correspondence Address: MCDERMOTT WILL & EMERY, LLP;34th Floor, 2049 Century Park East, Los Angeles, CA, 90067, US

(73) Assignee: Alfred E. Mann Institute for Biomedical Engineering at the University of S. California

(21) Appl. No.: 11/350,482

(22) Filed: Feb. 9, 2006

Related U.S. Application Data
(60) Provisional application No. 60/651,299, filed on Feb. 9, 2005.

(51) Int. Cl. A61N 1/18

(52) U.S. Cl. 607/48

(57) ABSTRACT

Disclosed are methods and systems for a virtual reality simulation and display of limb movement that facilitate the development and fitting of prosthetic control of a paralyzed or artificial limb. The user generates command signals that are then processed by the control system. The output of the control system drives a physics-based simulation of the limb that simulates the limb to be controlled. The computed movements of the model limb are displayed to the user as a 3D animation from the perspective of the user so as to give the impression that the user is watching the actual movements of his/her own limb. The user learns to adjust his/her command signals to perform tasks successfully with the virtual limb. Alternatively or additionally, the errors produced by the virtual limb and/or the responses of the user during the training process can provide information for adapting the properties of the control system itself.

### CROSS REFERENCE TO RELATED APPLICATIONS

This patent application is related to and claims the benefit of the filing date of U.S. provisional application Ser. No. 60/651,299, filed Feb. 9, 2005, entitled “Method and System for Training Adaptive Control of Limb Movement,” the contents of which are incorporated herein by reference.

### BACKGROUND OF THE INVENTION

1. Field of the Invention

The present invention relates generally to devices and methods to facilitate the development and fitting of prosthetic control of a paralyzed or artificial limb.

2. General Background and State of the Art

Patients with amputated or paralyzed limbs can be fitted with prosthetic systems to restore voluntary limb movement. Amputees use prosthetic limbs equipped with electrically controlled motors and clutches, hereafter referred to as “actuators”. Patients with paralysis as a result of spinal cord injury or stroke can be fitted with neuromuscular electrical stimulators to reanimate their own limbs. These are also actuators in our terminology. In both cases, the design and fitting of control algorithms for such prostheses tends to be difficult and time-consuming for all but the simplest functions.

### SUMMARY

Systems and methods for creating a virtual reality experience are based on a simulation of a neural prosthetic system for the control and generation of voluntary limb movement. Embodiments of the virtual reality systems and methods allow able-bodied subjects to experience the performance of such prosthetic systems in order to expedite their development and testing. The systems and methods facilitate the prescription, fitting and training of prosthetic systems in individual patients.

In one aspect of the virtual reality training methods and systems; a training system comprises a virtual reality display of limb movement in order to facilitate the development and fitting of a prosthetic and/or FES-enabled limb. The user generates command signals that are then processed by the control system. The output of the control system drives a physics-based model that simulates the limb to be controlled. The computed movements of the simulated limb are displayed to the user as a 3D animation from the perspective of the user so as to give the impression that the user is watching the actual movements of his/her own limb. The user learns to adjust his/her command signals to perform tasks successfully with the virtual limb. Alternatively or additionally, the errors produced by the virtual limb and/or the responses of the user during the training process can provide information for adapting the properties of the control system itself.

It is understood that other embodiments of the virtual reality limb training systems and methods will become readily apparent to those skilled in the art from the following detailed description, wherein it is shown and described only exemplary embodiments by way of illustration. As will be realized, the virtual reality limb training systems and methods are capable of other and different embodiments and its several details are capable of modification in various other respects. Accordingly, the drawings and detailed description are to be regarded as illustrative in nature and not as restrictive.

### BRIEF DESCRIPTION OF THE DRAWINGS

Aspects of the present invention are illustrated by way of example, and not by way of limitation, in the accompanying drawings, wherein:

FIG. 1 is an illustration of an exemplary embodiment of an adaptive limb training system;

FIG. 2 is a schematic diagram of another exemplary embodiment of an adaptive limb training system; and

FIG. 3 is a schematic diagram of an exemplary embodiment of an adaptive limb training method.

### DETAILED DESCRIPTION

The detailed description set forth below is intended as a description of exemplary embodiments of the virtual reality limb training systems and methods and is not intended to represent the only embodiments in which the virtual reality limb training systems and methods can be practiced. The term “exemplary” used throughout this description means “serving as an example, instance, or illustration,” and should not necessarily be construed as preferred or advantageous over other embodiments. The detailed description includes specific details for the purpose of providing a thorough understanding of the virtual reality limb training systems and methods. However, it will be apparent to those skilled in the art that the virtual reality limb training systems and methods may be practiced without these specific details. In some instances, well-known structures and devices are shown in block diagram form in order to avoid obscuring the concepts of the virtual reality limb training systems and methods.

Most patients will have residual voluntary control over some portions of the limb. Such voluntary movements can be sensed in order to provide command information about the patient's intended limb movements. In situations where the patient's capability for voluntary movement is insufficient to provide mechanical control signals, bioelectrical signals can be recorded from residual muscles under voluntary control or from the central nervous system itself, such as from motor cerebral cortex. The movements produced by the actuators can also be sensed in order to provide feedback information to adjust the control signals to the actuators in order to achieve the desired limb movement. The control system integrates these sources of command and feedback information to compute continuously the output to the various actuators according to a control algorithm. Because of the complexity of limb mechanics and differences in the condition and requirements of patients, it is frequently desirable to test the control algorithm on a computerized simulation of the prosthetic system rather than on the patients themselves. Such testing affords the opportunity to adjust the control algorithm either by direct intervention of an operator or by adaptive control, in which deviations of the simulated performance from the desired performance cause automatic changes in the control algorithm. It is also typically the case that the patient learns to adjust to imperfections in the behavior of the control algorithm by adapting his/her own strategies for generating command signals.

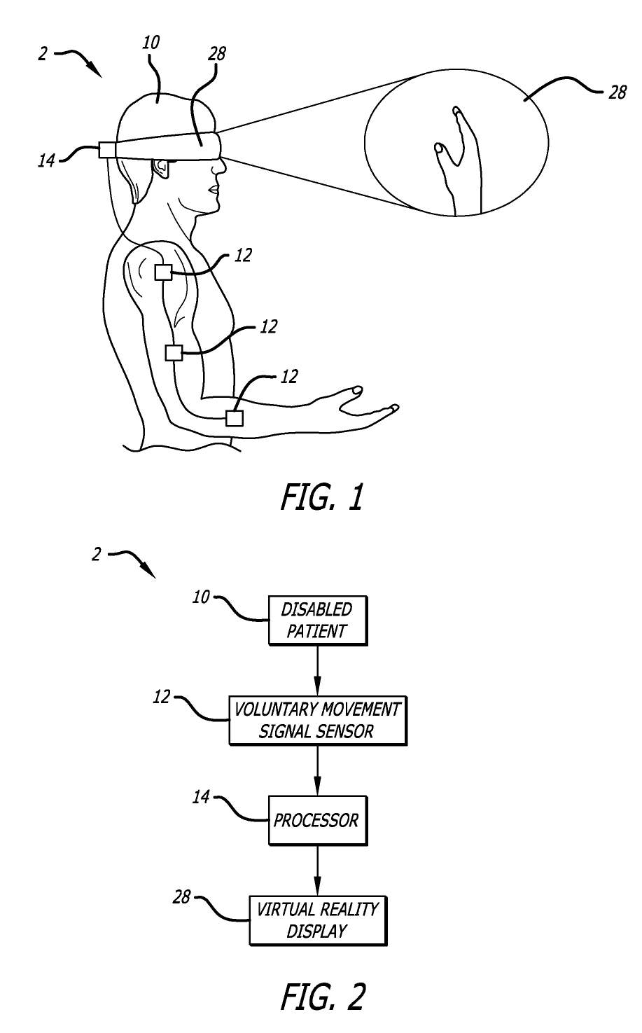

An adaptive limb modeling virtual reality system 2 is illustrated in FIGS. 1 and 2. A disabled patient 10 generates voluntary movement signals from an unimpaired portion of the patient's body. Signal sensors 12 sense the patient's 10 intended voluntary movement signal. The sensor 12 may be an EMG detector to detect residual muscle movements. Alternatively, it may be a sensor to detect signals from the central nervous system. For example, some embodiments may detect neural signals from peripheral motor neurons, while others may detect signals from the brain. A plurality of sensors 12 may be used to detect numerous intended limb movement signals. The sensor delivers the sensed signal to a processor 14, which determines the intended limb movement from the sensed signals and creates a dynamic simulation (discussed in detail below) of limb movement. The limb movement is animated and displayed to the patient 10 in a virtual reality environment via virtual reality display 28. The display 28 may be within a headpiece worn by the patient so that the patient experiences a virtual environment, as known to those skilled in the art. The patient can view the simulated limb movement, and adjust his intended voluntary limb movement commands to change the movement and position of the simulated limb.

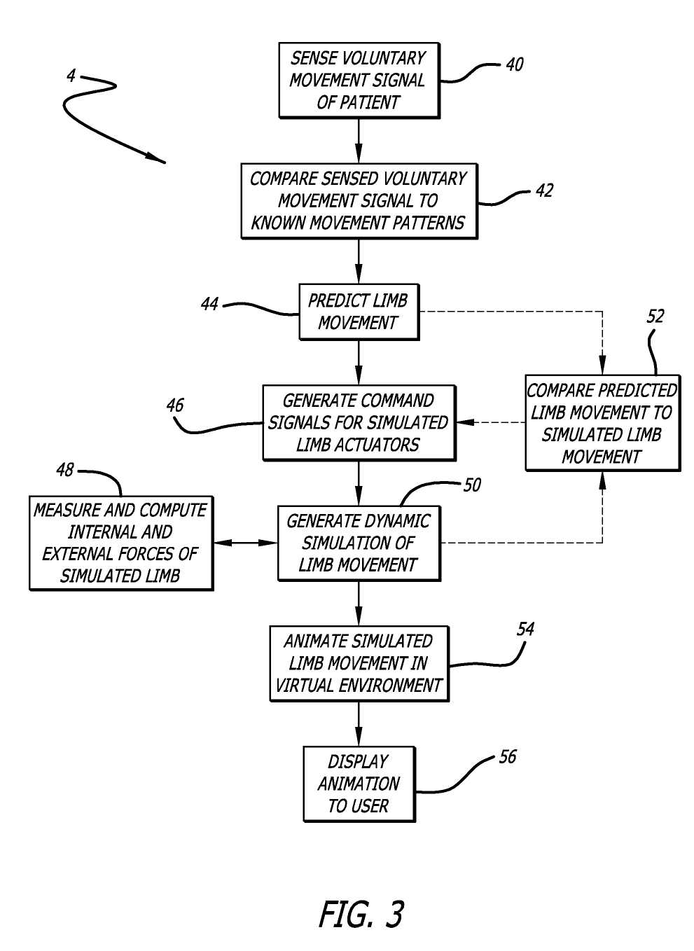

FIG. 3 schematically depicts an exemplary method of virtual reality training 4. First, the patient's voluntary movement signals are sensed 40 as discussed above. Then, the sensed voluntary movement signals are compared to known movement patters 42. This comparison of sensed signals to known patterns 42 can be achieved through a neural network, pattern recognition, or other method known to those skilled in the art. Then, the limb movement is predicted 44 based upon the sensed signal comparison 42. Based on the predicted limb movement 44, command signals are generated for simulated limb actuators 46. Then, a dynamic simulation of limb movement is generated 50 based on the command signals 46. The dynamic simulation also takes into account measured and computed internal and external forces of a simulated and/or actual limb 48. For example, such forces 48 can include numerous external forces (such as gravity) and internal forces of the limb (such as skeletal, muscular, joints, actuators, etc.) The simulated limb movement may then be animated 54 in a virtual environment. This animation 54 may be a computer-generated three dimensional (3-D) animation, as known to those skilled in the art. The animation 54 is then displayed 56 to the user. The displaying 56 can be achieved through a headpiece (as described in FIGS. 1 and 2 above).

In an exemplary embodiment also schematically illustrated in FIG. 3, the dynamic simulation of the movement of the simulated limb 50 is compared 52 to the predicted limb movement 44. The results of the comparison 52 (namely the discrepancy/error between the simulated limb movement 50 and the predicted limb movement 44) can be used to generate corrected command signals to control simulated limb actuators 46. This feedback mechanism can work in parallel with adjustments that the patient makes of his intended voluntary limb movement commands.

In an exemplary embodiment of the virtual reality limb training systems and methods, a method for a subject to control the movement of a virtual limb and experience virtual limb movement comprises initiating a movement in the limb by means of residual voluntary limb movement, measuring voluntary movements, inferring from a subset of the measured voluntary movements control signals to drive the prosthetic or paralyzed part of the limb, simulating the movement of the limb in response to control signals and other environmental forces, and displaying the animation of the simulated movement to the subject from his/her point of view. A control system can achieve the inferring of the movement of the rest of the limb. The measuring of voluntary movement collects data from motion sensors installed on the limb. The method can further comprise generating control signals, based upon data collected from said measuring voluntary movements, for actuators to produce the movement of the rest of the limb. Embodiments can further comprise predicting the movement trajectories caused by the actuators and other external influences such as gravity. A real-time computer program having a mathematical model of the neuromusculoskeletal properties of the rest of the limb can make such predictions. In some embodiments, the animating is based upon the measured and predicted joint trajectories. The display can be a stereoscopic display such as a head mounted display device. In some embodiments, when the subject successfully commands the simulated arm to move with the same trajectory as his/her intact arm, the subject can perceive similar sensory feedback as a patient would when operating the FES limb.

In another embodiment of the virtual reality limb training systems and methods, a system for training disabled patients control the movement of disabled joints with residual voluntary limb movement comprises motion sensors and actuators placed on the patient, and a processor, wherein the processor measures the patient's voluntary movements, identifies the patient's intended movement for the whole limb, generates control signals for the actuators on the limb to realize the patient's intended movement, predicts in real-time the movement trajectories caused by the actuators and other external influences such as gravity, and displays an animated virtual arm. In an exemplary embodiment, the motion sensors are installed on the intact joints. The actuators can be disabled insofar as to prevent them from causing limb movement. In some embodiments, the display can be a stereoscopic, head mounted display. Some embodiments can further provide sensory feedback to the patient. In such embodiments, when the subject successfully commands the simulated arm to move with the same trajectory as his/her intact arm, the subject can perceive similar sensory feedback as a patient would when operating the FES limb. In another embodiment, the control system parameters are designed off-line and kept constant during the operation while the patient's central nervous system adapts its behavior to match the predicted and intended movements. In yet another embodiment, the control system and the patient's central nervous system adaptively correct their behavior to eliminate the errors based upon the feedback of the errors between the predicted and desired movements of the disabled limb.

In yet another embodiment, a system for training disabled patients to control the movement of disabled joints with residual voluntary limb movement comprises motion sensors and actuators placed on the patient, and a processor, wherein the processor measures the patient's voluntary movements, identifies the patient's intended movement for the whole limb, and causes the actuators to move the limb according to the identified intended movement. In some embodiments, the motion sensors are installed on the intact joints. The system can further provide sensory feedback to the patient. In such embodiments, the patient will feel the movement of the disabled joints by the sensors in the intact part of the limb. The patient's central nervous system can use the sensory feedback and visual feedback of the limb movement to continue to adapt its behavior during the deployment phase.

In an exemplary embodiment of the present invention, the actuators and/or sensors can be implantable. For example, implantable microstimulators, methods and systems that can be used in some preferred embodiments of the present invention are disclosed in U.S. Pat. No. 5,324,316 (to Schulman et al.); U.S. Pat. No. 5,405,367 (to Schulman et al.); and U.S. Pat. No. 5,312,439 (to Loeb et al.); which are incorporated herein by reference.

In an exemplary embodiment, a head tracking device can be used to create a more realistic virtual environment. For example, an accelerometer can be positioned on the patient's head, such as on or in the display device, to sense the position of the patient's head. Therefore, when the patient looked away from his prosthetic or paralyzed limb, then the accelerometer would detect such movement and send a signal to the processor. The processor would then adjust the virtual reality simulation so that the virtual limb would not appear to the patient when the patient looks away from the location of the actual prosthetic or paralyzed limb.

In an exemplary device, the system can adjust the actuator control signals in response to results of the simulation. For example, if the simulated limb movement does not match the intended limb movement (as predicted from a pattern recognition program that can predict intended limb movement based upon information from the sensed intended movement signals of the patient), then the processor can adjust its movement command signals to the actual and/or simulated limb actuators. This can be a continuous process. Alternatively, the system may not adjust its command signals, so that the patient can adjust his intended voluntary movement signals to cause the limb to move as he intends. In yet another embodiment, the system provides for some adjustment in addition to allowing the patient to adjust his intended voluntary movement commands to cause the simulated limb to move as he wishes.

The virtual reality limb training systems and methods can allow subjects to study their ability to control a simulation of a paralyzed arm equipped with the FES interface. This is useful for control engineers to develop an intuitive feel for the strengths and weaknesses of the FES controllers that they intend to provide to patients. When using a controller operated by residual voluntary movement as described above, the operator needs to learn to make adjustments to those command movements in order to compensate for noise and errors in the FES system.

When the simulated movement that the intact subject sees in the virtual reality display matches the actual movement of the subject's intact limb, the subject will perceive the same motion and load in the muscles responsible for the command movements as the patient would feel when successfully performing the movement with the FES system. This is important because sensory feedback probably facilitates the ability of the operator to learn to use any control system. An FES system for control of reaching that uses the movement velocity of the upper arm to drive the FES control of the lower arm movement according to normal movement synergies is described in an article by Popovic and Popovic (D. Popovic and M. Popovic. Tuning of a nonanalytical hierarchical control system for reaching with FES. IEEE Trans. Biomed. Eng 45 (2):203-212, 1998), which is incorporated herein by reference. In another study, an FES system was developed in which the contralaterlal shoulder position was used to proportionally drive the electrically stimulated movement of the arm and hand. The control of hand grasp and release were coupled with stimulated arm motions so that hand-to-mouth activities could be accomplished with one motion of the contralateral shoulder. The system is described in article by Smith et al. (B. T. Smith, M. J. Mulcahey, and R. R. Betz. Development of an upper extremity FES system for individuals with C4 tetraplegia. IEEE Trans. Rehabil. Eng 4 (4):264-270, 1996) which is incorporated herein by reference.

In an exemplary embodiment, the virtual reality limb training systems and methods create dynamic limb simulations. The purpose of dynamic simulation is to calculate the realistic movement of the paralyzed or artificial limb in response to control inputs and external forces. An exemplary embodiment incorporates properties of the limb components such as segments, joints, and actuators to model the limb. In addition, the force of gravity on various portions of the limb may also be taken into account. Then, principles such as Newton's laws of motion are applied to the model to derive the set of equations that govern the movement of the limb. The solution of these equations over time then predicts the motion of the limb in response to control inputs and external forces. Therefore, for any given control strategy, the system can predict the realistic movement of the limb and display it to the subject as an indication of the movement they would experience if they had really worn the prosthetic arm. For example, force equations for various forces (such as those described above) can be integrated to obtain acceleration values. The acceleration values could then be integrated to obtain velocity values. Velocity values could then be integrated to obtain position values over various times. Such calculations can occur continuously over time to determine what the position of components of the limb, and position of the limb itself, would be at numerous times.

Movement of the human limb is the result of complicated interactions involving voluntary command signals, sensory receptors, reflex circuits, muscle actuators, the skeleton, gravity, and the environment. Design of controllers for such complex system is a difficult task and typically cannot be accomplished by trial and error on the patient. The computer models can play the role of a virtual limb with precisely controllable experimental conditions for the design and evaluation of controllers prior to human trials. Stability and behavior of the system under various conditions, and sensitivity to variations in the model and control system parameters, can be investigated. The following articles, which are incorporated by reference, provide examples of dynamic limb models that can be used in some embodiments: R. Davoodi and B. J. Andrews. Computer simulation of FES standing up in paraplegia: a self-adaptive fuzzy controller with reinforcement learning. IEEE Trans. Rehabil. Eng 6 (2):151-161, 1998; M. A. Lemay and P. E. Crago. A dynamic model for simulating movements of the elbow, forearm, an wrist. J. Biomech. 29 (10):1319-1330, 1996; and G. T. Yamaguchi and F. E. Zajac. Restoring unassisted natural gait to paraplegics via functional neuromuscular stimulation: a computer simulation study. IEEE Trans. Biomed. Eng 37 (9):886-902, 1990.

In another embodiment, the virtual reality adaptive training system can be used simultaneously with a functioning prosthetic limb or stimulators implanted in a paralyzed limb. In yet another exemplary embodiment, the patient may receive somatosensory feedback of limb movement in addition to visual feedback from the virtual reality display.

The previous description of the disclosed embodiments is provided to enable any person skilled in the art to make or use the virtual reality systems and methods. Various modifications to these embodiments will be readily apparent to those skilled in the art, and the generic principles defined herein may be applied to other embodiments without departing from the spirit or scope of the virtual reality systems and methods. Thus, the virtual reality systems and methods are not intended to be limited to the embodiments shown herein but are to be accorded the widest scope consistent with the principles and novel features disclosed herein.

What is claimed is:

1) A training system that displays to a patient simulated movements of a virtual limb comprising:
a) at least one sensor configured to sense a patient's voluntary movement signals from an unimpaired portion of the patient's body and deliver the sensed signal to a processing system;
b) a processing system configured to:
i) receive the sensed voluntary movement signals from the at least one sensor;
ii) predict the intended limb movement;
iii) generate command signals to control simulated limb actuators based on the predicted limb movement; and
iv) create a dynamic simulation of limb movement based on the simulated limb actuators, and a plurality of internal and external forces of a simulated limb; and
c) a display device configured to communicate with the processing system and display animation of the simulated movements of the simulated limb to the patient in a virtual environment.

2) The training system of claim 1, wherein at least one of the forces is gravity.

3) The training system of claim 1, wherein the animation is 3D animation.

4) The training system of claim 3, wherein the display device is mounted on the patient's head.

5) The system of claim 1, wherein the display device further comprises a head motion-tracking device.

6) The system of claim 1, wherein the processing system is further configured to compare the predicted limb movement to the simulated limb movement.

7) The system of claim 6, wherein the processing system is further configured to adjust its command signals to control the simulated limb actuators so that the simulated limb movement matches the predicted intended limb movement.

8) The system of claim 1, wherein the at least one sensor is configured to sense cortical signals.

9) The system of claim 1, wherein the at least one sensor is configured to sense residual voluntary muscle movement.

10) The system of claim 1, wherein the at least one sensor is an implantable microstimulator.

11) The system of claim 1, wherein the processing system is configured to analyze the sensed voluntary movement signals to determine whether it matches a known movement pattern.

12) A processing system configured to:
a) receive a sensed voluntary movement signal from a patient sensor;
b) predict intended limb movement based upon the sensed voluntary movement signal;
c) generate command signals to control simulated limb actuators based on the predicted limb movement; and
d) create a dynamic simulation of limb movement based on the simulated limb actuators, and a plurality of internal and external forces of a simulated limb.
